(19) United States
(12) Patent Application Publication
(10) Pub. No.: US 2013/0236396 A1
(43) Pub. Date: Sep. 12, 2013

(54) NANOPARTICLES PRODUCED FROM RECOMBINANT POLYMERS AND METHODS OF MAKING AND USING THE SAME

(71) Applicants: Leonard Franklin Pease, III, Bountiful, UT (US); Hamid Ghandehari, Salt Lake City, UT (US); Rajasekhar Anumolu, Salt Lake City, UT (US)

(72) Inventors: Leonard Franklin Pease, III, Bountiful, UT (US); Hamid Ghandehari, Salt Lake City, UT (US); Rajasekhar Anumolu, Salt Lake City, UT (US)

(73) Assignee: UNIVERSITY OF UTAH RESEARCH FOUNDATION, Salt Lake City, UT (US)

(21) Appl. No.: 13/695,827

(22) PCT Filed: May 3, 2011
(86) PCT No.: PCT/US11/34907

§ 371 Date: Mar. 20, 2013

Related U.S. Application Data
(60) Provisional application No. 61/343,758, filed on May 3, 2010.
(60) Provisional application No. 61/456,633, filed on Nov. 9, 2010.

(51) Int. Cl. A61K 47/42

(52) U.S. Cl. CPC A61K 47/42
USPC 424/9.1; 530/353; 428/402; 514/773; 514/44 R; 514/1.1; 424/130.1; 424/93.6

(57) ABSTRACT

Described herein are nanoparticles produced from recombinant polymers. The nanoparticles are substantially uniform in size, which provides numerous advantages with respect to the delivery of bioactive agents to a subject. Methods for making the nanoparticles are also described herein. In one aspect, the nanoparticles are produced by the method comprising: a. providing a solution comprising one or more recombinant polymer in a solvent; b. forming droplets comprising the one or more recombinant polymers and the solvent; c. removing the solvent to produce the nanoparticles; and d. separating the nanoparticles based on size to produce nanoparticles that are substantially uniform in size. Finally, pharmaceutical compositions composed of the nanoparticles and methods of using the same are also described.

### CROSS REFERENCE TO RELATED APPLICATION

This application claims priority upon U.S. Provisional Application Serial Nos. 61/343,758, filed May 3, 2010, and 61/456,633, filed on Nov. 9, 2010. These applications are hereby incorporated by reference in their entireties for all of their teachings.

### ACKNOWLEDGEMENTS

The research leading to this invention was funded in part by the National Institutes of Health, Grant No. R01CA107621. The U.S. Government has certain rights in this invention.

### BACKGROUND

Nanoparticles are widely used for a variety of biomedical applications including targeted drug and gene delivery. However, most nanoparticles with refined size are composed of metallic particles with potential toxicity issues (e.g., quantum dots, silver particles, etc.). In response to this, polymeric matrices have been used for localized gene delivery. Despite some success using polymeric matrices, they have primarily been used for direct injection into tissues such as solid tumors, which due to poor accessibility and patient inconvenience can limit the broader application of these polymers. Described herein are the preparation and use of nanoparticles produced from recombinant polymers that can be administered systemically for the delivery of bioactive agents to the target site. The ability to deliver highly uniform nanoparticles systemically permits the nanoparticles to be used in a variety of different applications with improved functions such as reduced toxicity and maximized efficacy.

### SUMMARY

Described herein are nanoparticles produced from recombinant polymers. The nanoparticles are substantially uniform in size, which provides numerous advantages with respect to the delivery of bioactive agents to a subject. Methods for making the nanoparticles are also described herein. In one aspect, the nanoparticles are produced by the method comprising:

a. providing a solution comprising one or more recombinant polymer in a solvent;
b. forming droplets comprising the one or more recombinant polymers and the solvent;
c. removing the solvent to produce the nanoparticles; and
d. separating the nanoparticles based on size to produce nanoparticles that are substantially uniform in size.
Finally, pharmaceutical compositions composed of the nanoparticles and methods of using the same are also described.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows (a) a schematic of electrospray differential mobility analysis (ES-DMA) including electrospray (ES) to generate highly charged droplets enclosing multiple polymer strands; a neutralizer to set the charge on the drying nanoparticles to +1, −1, or 0; a differential mobility analyzer (DMA) to separate particles by their charge-to-size ratio determined trajectory by balancing electrostatic, F_{E} and drag forces, F_{D}; and a condensation particle counter (CPC) to enumerate them or an aerosol sampler (ED) to deposit them on desired substrates. The magnified droplet depicts the nanoparticle formation process in which the individual polymer strands entangle as the droplet evaporates. (b) A gallery of TEM images of representative SELP nanoparticles.

FIG. 2 shows (a) size distributions of nanoparticles fabricated from polymers SELP-815K (▴), SELP-415K (▪), and SELP-47K (♦) at polymer weight fraction and buffer concentration of wₚ=0.00133 and C_{b}=2 mM, respectively. Number density is the number of particles/cc. The insets show micrographs of SELP-815K nanoparticles electrostatically collected on TEM grids (dark line) from peaks in the size distribution at 24.0 nm and 36.0 nm, respectively, to demonstrate the size selectivity of the DMA. (b) Histograms representing the diameter of SELP-815K nanoparticles as determined from TEM following electrostatic deposition of nominally 24.0 nm and 36.0 nm. The mean and standard deviation of these particles are 24.2±1.2 nm and 35.8±1.4 nm, respectively.

FIG. 3 shows (a) experimental peak mobility diameter, dₚ, (see FIG. 2a) versus polymer concentration, wₚ, to the ⅓ power for SELP-415K (♦), SELP-47K (▪), and SELP-815K (▴) at C_{b}=2 mM. (b) The Mobility diameter versus buffer concentration, C_{b}, to the −⅓ power at wₚ=0.00133.

FIG. 4 shows (a) a TEM micrograph depicting the length of a facet, L_{f}, on a 36 nm diameter SELP nanoparticle with d_{b}˜3-4 nm. (b) Diagram showing the three stages of SELP nanoparticle growth, namely, (i) evaporation of an electrospray droplet containing polymer strands, (ii) accumulation and entanglement of the strands at the droplet surface until a thin film gels to form a shell of thickness h, and (iii) buckling of the shell to relieve compression energy, F_{c}, by bending to reveal the facets of panel (a). (c) Ratio of the bend diameter, d_{b}, to the length of the facet versus the ratio of the shell thickness to the particle diameter, dₚ. The solid lines represent Eq. 2, the symbols represent experimental data, and the numbers to the right represent the number of facets. (d) The mean and standard deviation (as error bars) of the equivalent diameter (right bar) and facet length (left bar) following electrostatic deposition at two nominal sizes for each of the three polymers in Table 1.

FIG. 5 shows (a) growth of instability with characteristic period and radius of λ and Lᵥ on an electrospray jet of diameter dⱼₑₜ. (b) Ratio of the characteristic radius of the instability to the diameter of the jet versus the polymer concentration for κ˜1.238 S/m (▪) (C_{b}˜45 mM and d_{drop}=100 nm), κ√0.303 S/m (♦) (C_{b}˜11 mM and d_{drop}=200 nm), and κ√0.028 S/m (▴) (C_{b}˜0.2 mM and d_{drop}=300 nm). The jet breaks up into droplets for 2Lᵥ/dⱼₑₜ<1 and remains as a thread or rod-like structures for 2Lᵥ/dⱼₑₜ˜>1. Mostly spherical and some rod-like structures are formed at wₚ=0.00133 and C_{b}=2 mM as shown in panels i, ii, and iii, due to uncertainty in dⱼₑₜ.

FIG. 6 shows the size distribution of SELP-815K polymer (▪) at a concentration of wₚ=0.00066 in ammonium acetate buffer at C_{b}=2 mM mixed with (a) GFP labeled DNA plasmids (103 μg/mL) (♦) and (b) fluorescein isothiocyanate (FITC, 1.8 mg/mL) (♦).

FIG. 7 shows the amino acid sequences of SELP-47K, SELP-415K, and SELP-815K.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

The term “silk-like” units as used herein have the amino acid sequences GAGAGS or SGAGAG.

The term “elastin-like” units as used herein have the amino acid sequence VPGG, APGVGV, GXGVP or VPGXG, where X is valine, lysine, histidine, glutamic acid, arginine, aspartic acid, serine, tryptophan, tyrosine, phenylalanine, leucine, glutamine, asparagine, cysteine or methionine, usually valine or lysine.

The term “collagen-like” units contain the tandemly repeated amino acid triad GXO, where G is glycine and X and O are any amino acid such as, for example, alanine, isoleucine, valine, leucine, serine, threonine, asparagine, glutamine, lysine, arginine, aspartic acid, glutamic acid, histidine or proline.

The term “keratin-like” units as used herein have “heptad” repeat unit composed of a seven amino acid long stretch with two positions separated by two amino acids, usually positions three and six, occupied consistently with hydrophobic, aliphatic or aromatic residues, e.g., AKLKLAE or AKLELAE.

The term “targeting ligand” as used herein includes any molecular signal directing localization to specific cells, tissues, or organs. Proteins that bind to cell surface receptors come within the definition of targeting ligand as do antibodies directed to antigens expressed selectively on a target cell.

The term “nuclear localization signal” (NLS) as used herein means any compound capable of facilitating the active nuclear import and/or export of proteins from the nucleus. Typically NLS are amino-acid sequences, often having basic amino acids. Any protein or peptide facilitating the active nuclear import and/or export of proteins is an NLS for the purposes described herein.

The term “endosome disrupting moiety as used herein means any protein or peptide capable of disrupting or lysing the endosome membrane resulting in release of the endosomal content; it is usually a sequence of amino acids.

The term “target cells” as used herein means any eukaryotic or prokaryotic cell intended as the recipient cell for delivery of a bioactive agent, including any animal cell whether normal or diseased such as a cancer cell, bacterial and plant cells.

Described herein are nanoparticles produced from recombinant polymers. The nanoparticles are substantially uniform in size, which provides numerous advantages with respect to the delivery of bioactive agents to a subject. Methods for making the nanoparticles are also described herein. In one aspect, the nanoparticles are produced by the method comprising:

a. providing a solution comprising one or more recombinant polymer in a solvent;
b. forming droplets comprising the one or more recombinant polymers and the solvent;
c. removing the solvent to produce the nanoparticles; and
d. separating the nanoparticles based on size to produce nanoparticles that are substantially uniform in size.
Each step and component used to produce the nanoparticles are described in detail below.

### I. Recombinant Polymers

The nanoparticles described herein are produced by one or more recombinant polymers. The recombinant polymers useful herein can be genetically engineered proteins composed of multiple repeating amino acid residues. The protein can be transcribed from a single gene, where the sequence can be found anywhere in nature or, in the alternative, can be an artificial sequence. The recombinant polymer is composed of amino acids which are arranged in a sequential manner within a block or set of blocks that are repeated in tandem producing a high molecular weight repetitive polymer. Recombinant polymer synthesis allows the systematic correlation of polymer structure with function, thus enabling customization to suit specific delivery needs.

Numerous recombinant polymers known in the art can be used herein. The recombinant polymers can be composed of a number of different amino acid motifs, where the number and order of the motifs can be specifically designed depending upon the application of the nanoparticles. For example, the recombinant polymer can be composed of silk-like units, elastin-like units, collagen-like units, keratin-like units, or any combination thereof. Any of the recombinant polymers disclosed and produced in U.S. Published Application Nos. 2007/0098702, 2010/0022455, and 2009/0093621, which are incorporated by reference, can be used herein.

In one aspect, the recombinant polymer comprises one or more silk-elastinlike protein polymers (SELP). In this aspect, the SELP is composed of silk-like and elastin-like units as defined herein in a specific order. The number of silk-like and elastin-like units can vary depending upon the application of the nanoparticles. In one aspect, the ratio of silk-like units to elastin-like units present is from 1:20 to 20:1. In other aspects, the ratio is 1:2 to 1:10, 1:2 to 1:8, 1:2 to 1:6, or 1:2 to 1:4.

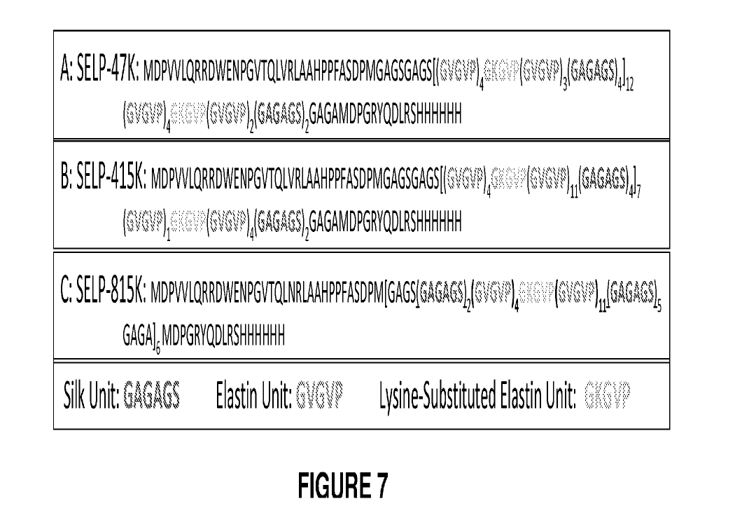

In another aspect, the SELP has a molecular weight from 25 kDa to 200,000 kDa. In a further aspect, the SELP has 2 to 10 silk units and 2 to 20 elastin units. In another aspect, the SELP is SELP-47K (SEQ ID NO. 1), SELP-415K SEQ ID NO. 2, SELP-815K (SEQ ID NO. 3), or any combination thereof. The amino acid sequences SELP-47K, SELP-415K, and SELP-815K are provided in FIG. 7. Methods for preparing SEQ ID NOS. 1-3 are disclosed in Ghandehari et al., Polymer 2009, 50, 366-374, which are incorporated by reference. In other aspects, the SELPs disclosed in U.S. Published Application Nos. 2010/143487, 20100261652, and 2009/0093621, which are incorporated by reference, can be used herein.

In certain aspects, the recombinant polymer includes a residue of at least one cationic amino acid. Examples of cationic amino acids include lysine, arginine, histidine, and cysteine. Lysine and arginine are cationic amino acids that are positively charged at pH 7.4, thus enabling them to bind negatively charged bioactive agents (e.g., nucleic acids) for delivery to a target cell. The cationic amino acid histidine (H) is not positively charged at pH 7.4 because the pKa of histidine is about 6. However, histidine is often included because it disrupts endosomes facilitating the release of the nucleic acid/vector complex from the endosome. Additionally, cationic amino acids like lysine permit the modification of the recombinant polymer. For example, bioactive agents and other functional compounds can be attached to the backbone of the recombinant polymer.

In certain aspects, the recombinant polymers can include one or more optional specialized moieties such as targeting ligands, endosome disrupting moieties, and nuclear localization sequences that facilitate the administration of the nanoparticles to a subject (e.g., systemically). Any of the targeting ligands, endosome disrupting moieties, and nuclear localization sequences disclosed in U.S. Published Application No. 2007/0098702, which is incorporated by reference, can be used herein.

The targeting ligand can be any amino acid based sequence that selectively targets a particular cell in order to facilitate the delivery of the nanoparticle with bioactive agent to that particular cell. Such motifs can target any cell surface receptor such as growth factor receptors (e.g., fibroblast growth factor, epidermal growth factor, etc.) or hormone receptors. Specific cell surface antigens can also be targeted using a complementary antibody. An example of a targeting ligand is FGF2, which comes in high and low molecular weight forms. High molecular weight FGF2 (HMW-FGF2) is a protein of 22, 22.5 or 24 kDa that contains a nuclear localization signal. Low molecular (LMW-FGF2) (17.5 kDa) does not have an NLS. Thus HMW-FGF2 is a multipurpose moiety, where it is a targeting ligand that provides NLS.

The recombinant polymer can contain a region that disrupts endosomes typically by lysing the endosome membrane. In certain aspects, direct gene delivery to the cytoplasm using electroporation or nucleus by the nanoparticles described herein may be desired and so there would be no need for an endosome disrupting moiety (EDM). Endosome lysis can be accomplished by using recombinant polymers having a polymer region that is rich in histidine. The optimum ratio of histidine to other amino acids in the polymer varies depending on the composition of the final construct and on the specific nucleic acid or therapeutic oligonucleotide intended for delivery.

In other aspects, the recombinant polymer includes an NLS to direct the bioactive agent (e.g., nucleic acid) to the nucleus where it is transcribed by the target cell. Any amino acid sequence that enhances the nuclear targeting of the nanoparticles composed of the bioactive agent can be considered a nuclear localization signal. An example of a known NLS that can be used in the vectors of the present invention comes from the Simian Virus SV40 large tumor antigen; the NLS comprises a single short stretch of basic amino acids (PKKKRKV) or (PNKKKRK). Other examples of NLS sequences are (RLRFRKPKSKD) in Feline Immunodeficiency Virus, (RRKRQR) in Dorsal protein, (KRRR) in adenovirus adenain protein, (RKRKR) in OCT4 protein, (RQARRNRRRRWRERQRQ) in Human Immunodeficiency Virus type 1 (HIV-1), (KSKKQK) in chicken v-rel protein, (KTRKHRG) in Ribosomal L29 protein, (GKKRSKAK) in yeast histone 2b, and (PVKKRKRK) in Rac1 protein.

Some known NLS sequences are bipartite having two stretches of basic amino acids separated by a spacer, such as is illustrated below. These include (KR-11 as spacer-KKLR) in RB protein; (RKKRK-12 aa spacer-KKSK) in N1N2 protein; (KKR-11aa spacer-KRVR) in adeno-associated virus Rep68/78 protein; (KRKGDEVDGVDEVAKKKSKK) in Poly(ADP-ribose)polymerase; (KRPMNAFIVWSRDQRRK) in Human SRY protein; (RLRRDAGGRGGVYEHLLGGAPRRRK) in Mouse FGF3; and (KRPAATKKAGQAKKKKL) in Xenopus nucleoplasmin protein.

Other known NLS sequences have charged/polar residues interspersed with non-polar residues such as the NLS [MNKIPIKDLLNPQ] in the yeast homeodomain containing protein Mat-α-2. Examples of NLS that target import in Beta include: (LGDRGRGRALPGGRLGGRGRGRAPERVGGRGRGRGTRAARGSRPGPAGTM) in high molecular weight basic fibroblast growth factor, amino acids 427-455 in Regulatory Factor X Complex; (SANKVTKNKSNSSPYLNKRKGKPGPDS) in Pho4; (VHSHKKKKIRTSPTFTTPKTLRLRRQKYPRKSAPRRNKLDHY) in rpL23a protein; and (MAPSAKATAAKKAVVKGTNGKKALKVRTSATFRLPKTLKLAR) in rpL25 protein.

The nanoparticles described herein are generally used to deliver a bioactive agent to a target cell. In one aspect, the bioactive agent comprises, a natural or synthetic oligonucleotide, a natural or modified/blocked nucleotide/nucleoside, a nucleic acid, a peptide comprising natural or modified/blocked amino acid, an antibody or fragment thereof, a hapten, a biological ligand, a virus, a membrane protein, a lipid membrane, an imaging agent, or a small pharmaceutical molecule.

In one aspect, the bioactive agent can be a protein. For example, the protein can include peptides, fragments of proteins or peptides, membrane-bound proteins, or nuclear proteins. The protein can be of any length, and can include one or more amino acids or variants thereof. The protein(s) can be fragmented, such as by protease digestion, prior to analysis. A protein sample to be analyzed can also be subjected to fractionation or separation to reduce the complexity of the samples. Fragmentation and fractionation can also be used together in the same assay. Such fragmentation and fractionation can simplify and extend the analysis of the proteins.

In one aspect, the bioactive agent is a virus. Examples of viruses include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, Vaccinia virus, SARS virus, Human Immunodeficiency virus type-2, lentivirus, baculovirus, adeno-associated virus, or any strain or variant thereof.

In another aspect, the bioactive agent can be an imaging agent. The term “imaging agent” is defined herein as any agent or compound that increases or enhances the ability of cells or tissues to be imaged or viewed using techniques known in the art when compared to visualizing the cells or tissue without the imaging agent. The imaging agent can be covalently or non-covalently attached to the nanoparticle.

In one aspect, a chelating agent is covalently attached to the recombinant polymer prior to nanoparticle formation. A chelating agent is any agent that can form non-covalent bond (e.g., complexation, electrostatic, ionic, dipole-dipole, Lewis acid/base interaction) with the imaging agent. The chelating agent can possess a group that can react with one or more groups on the recombinant polymer to form a covalent bond. For example, an amino group present on the recombinant protein can react with a carboxylic group on the chelating agent to produce an amide bond.

A number of different chelating agents known in the art can be used herein. In one aspect, the chelating agent comprises an acyclic or cyclic compound comprising at least one heteroatom (e.g., oxygen, nitrogen, sulfur, phosphorous) that has lone-pair electrons capable of coordinating with the imaging agent. An example of an acyclic chelating agent includes ethylenediamine. Examples of cyclic chelating agents include diethylenetriaminepentaacetate (DTPA) or its derivatives, 1,4,7,10-tetraazadodecanetetraacetate (DOTA) and its derivatives, 1,4,7,10-tetraazadodecane-1,4,7-triacetate (DO3A) and its derivatives, ethylenediaminetetraacetate (EDTA) and its derivatives, 1,4,7,10-tetraazacyclotridecanetetraacetic acid (TRITA) and its derivatives, 1,4,8,11-tetraazacyclotetradecane-1,4,8,11-tetraacetic acid (TETA) and its derivatives, 1,4,7,10-tetraazadodecanetetramethylacetate (DOTMA) and its derivatives, 1,4,7,10-tetraazadodecane-1,4,7-trimethylacetate (DO3MA) and its derivatives, N,N′,N″,N′″-tetraphosphonatomethyl-1,4,7,10-tetraazacyclododecane (DOTP) and its derivatives, 1,4,7,10-tetraazacyclododecane-1,4,7,10-tetrakis(methylene methylphosphonic acid) (DOTMP) and its derivatives, 1,4,7,10-tetraazacyclododecane-1,4,7,10-tetrakis(methylene phenylphosphonic acid) (DOTPP) and its derivatives. The term “derivative” is defined herein as the corresponding salt and ester thereof of the chelating agent.

Imaging agents known in the art can be used herein. In one aspect, the imaging agent comprises a denoptical dye, a MRI contrast agent, a PET probe, a SPECT probe, a CT contrast agent, or an ultrasound contrast agent. In one aspect, imaging agents useful in magnetic resonance imaging include Gd⁺³, Eu⁺³, Tm⁺³, Dy⁺³, Yb⁺³, Mn⁺², or Fe⁺³ ions or complexes. In another aspect, imaging agents useful in PET and SPECT imaging include ⁵⁵Co, ⁶⁴Cu, ⁶⁷Cu, ₄₇Sc, ⁶⁶Ga, ⁶⁸Ga, ⁹⁰Y, ⁹⁷Ru, ^{99m}Tc, ₁₁₁In, ¹⁰⁹Pd, ¹⁵³Sm, ¹⁷⁷Lu, ¹⁸⁶Re, ¹⁸⁸Re. The complexing of the imaging agent to the recombinant polymer having one or more chelating agents can be performed using routine techniques. For example, a salt of the imaging agent can be dissolved in a solvent and admixed with the recombinant polymer prior to the nanoparticles formation.

In another aspect, the bioactive agent is a nucleic acid. The nucleic acid can be an oligonucleotide, deoxyribonucleic acid (DNA), ribonucleic acid (RNA), or peptide nucleic acid (PNA). The nucleic acid of interest introduced by the present method can be nucleic acid from any source, such as a nucleic acid obtained from cells in which it occurs in nature, recombinantly produced nucleic acid, or chemically synthesized nucleic acid. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In one aspect, the nucleic acid can be present in a vector such as an expression vector (e.g., a plasmid or viral-based vector). In another aspect, the nucleic acid selected can be introduced into cells in such a manner that it becomes integrated into genomic DNA and is expressed or remains extrachromosomal (i.e., is expressed episomally). In another aspect, the vector is a chromosomally integrated vector. The nucleic acids useful herein can be linear or circular and can be of any size with the provision that when condensed they are smaller than the dry external dimensions of the nanoparticle. In one aspect, the nucleic acid can be single or double stranded DNA or RNA.

In one aspect, the nucleic acid can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, siRNA, miRNA, shRNA and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acids can be a small gene fragment that encodes dominant-acting synthetic genetic elements (SGEs), e.g., molecules that interfere with the function of genes from which they are derived (antagonists) or that are dominant constitutively active fragments (agonists) of such genes. SGEs can include, but are not limited to, polypeptides, inhibitory antisense RNA molecules, ribozymes, nucleic acid decoys, and small peptides. The small gene fragments and SGE libraries disclosed in U.S. Patent Publication No. 2003/0228601, which is incorporated by reference, can be used herein.

The functional nucleic acids of the present method can function to inhibit the function of an endogenous gene at the level of nucleic acids, e.g., by an antisense, RNAi or decoy mechanism. Alternatively, certain functional nucleic acids can function to potentiate (including mimicking) the function of an endogenous gene by encoding a polypeptide that retains at least a portion of the bioactivity of the corresponding endogenous gene, and may in particular instances be constitutively active.

Other therapeutically important nucleic acids include antisense polynucleotide sequences useful in eliminating or reducing the production of a gene product, as described by Tso, P. et al Annals New York Acad. Sci. 570:220-241 (1987). Also contemplated is the delivery of ribozymes. These antisense nucleic acids or ribozymes can be expressed (replicated) in the transfected cells. Therapeutic polynucleotides useful herein can also code for immunity-conferring polypeptides, which can act as endogenous immunogens to provoke a humoral or cellular response, or both. The polynucleotides employed according to the present invention can also code for an antibody. In this regard, the term “antibody” encompasses whole immunoglobulin of any class, chimeric antibodies and hybrid antibodies with dual or multiple antigen or epitope specificities, and fragments, such as F(ab)₂, Fab², Fab and the like, including hybrid fragments. Also included within the meaning of “antibody” are conjugates of such fragments, and so-called antigen binding proteins (single chain antibodies) as described, for example, in U.S. Pat. No. 4,704,692, the contents of which are hereby incorporated by reference.

In one aspect, the nucleic acid is siRNA. siRNAs are double stranded RNA molecules (dsRNAs) with approximately 20 to 25 nucleotides, which are generated by the cytoplasmic cleavage of long RNA with the RNase III enzyme Dicer. siRNAs specifically incorporate into the RNA-induced silencing complex (RISC) and then guide the RNAi machinery to destroy the target mRNA containing the complementary sequences. Since RNAi is based on nucleotide base-pairing interactions, it can be tailored to target any gene of interest, rendering siRNA an ideal tool for treating diseases with gene silencing. Gene silencing with siRNAs has a great potential for the treatment of human diseases as a new therapeutic modality. Numerous siRNAs have been designed and reported for various therapeutic purposes and some of the siRNAs have demonstrated specific and effective silencing of genes related to human diseases. Therapeutic applications of siRNAs include, but are not limited to, inhibition of viral gene expression and replication in antiviral therapy, anti-angiogenic therapy of ocular diseases, treatment of autoimmune diseases and neurological disorders, and anticancer therapy. Therapeutic gene silencing has been demonstrated in mammals, which bodes well for the clinical application of siRNA. It is believed that siRNA can target every gene in human genome and has unlimited potential to treat human disease with RNAi.

### II. Preparation of Nanoparticles

The first step for preparing the nanoparticles described herein involves preparing a solution of the recombinant polymer and bioactive agent. In general, the solvent used is not toxic or does not present safety or environmental concerns. The solvent also should be easily evaporated, preferably easier to evaporate than water. In certain aspects, a co-solvent such as, for example, an alcohol like methanol or ethanol can be used in combination with water to enhance the evaporation rate of the solvent. Thus, the solvent can be water alone or in combination with other co-solvents. In another aspect, the solvent is a buffered solution. In certain aspects, it is desirable that the buffered solution have an intermediate ionic strength in techniques such as, for example, electrospray differential mobility analysis (ES-DMA). In one aspect, the buffered solution is composed of a volatile salt including, but not limited to, ammonium salts. An example of an ammonium salt useful herein includes ammonium acetate. The concentration of the buffered solution can range from 0.02 mM to 100 mM, 0.1 mM to 100 mM, 0.5 mM to 50 mM, 1 mM to 30, or 2 mM to 20 mM. In other aspects, the solution can include acids such as, for example, glacial acetic acid or formic acid. The concentration of the acid can vary depending upon the ionic strength of the acid and the desired level of conductivity.

In general, the recombinant polymers are hydrophilic and, thus water soluble. By selecting the number and order of amino acid motifs in the polymer, it is possible to modify the hydrophilic properties of the polymer. In the case of SELPs, the nature of the elastinlike blocks, and their length and position within the monomers influences the water solubility of the SELP. For example, decreasing the length and/or content of the silk-like block domains, while maintaining the length of the elastin-like block domains, increases the water solubility of the polymers. The concentration of the recombinant polymer can vary as well. In one aspect, the recombinant polymer is from 0.0005 wt % to 0.5 wt % of the composition. The concentration of the recombinant polymer and buffer influence the size distribution of the nanoparticles that are produced. By selectively modifying these parameters it is possible to tune the yield of particles at a particular size. For example, as shown in the Examples, increasing the concentration of the recombinant polymer results in the formation of larger particles. Conversely, increasing the concentration of the buffer produces smaller nanoparticles.

After the solution composed of the recombinant polymer and bioactive agent has been produced, the solution is subjected to conditions in which the solution is converted to an aerosol. In one aspect, a nebulizer or electrospray aerosol generator can be used. In the case of electrospray ionization, strands of the recombinant polymer are aerosolized into droplets, which are then entrained in a stream of nitrogen at atmospheric pressure. In one aspect, the droplets span 150 nm to 300 nm in diameter though larger sizes are possible, and several strands may reside within each droplet. The number of strands within the droplet can be tuned by controlling the product of droplet volume as well as the polymer and buffer concentration in solution. Once the droplet has formed it is electrostatically stabilized by passing it through a charge neutralizer at atmospheric pressure to reduce the net charge on the aerosolized droplet. This prevents the Rayleigh instability that fragments molecules in ES-MS instruments. In the charge neutralizer, the droplet also dries. As the solvent evaporates the concentration of the polymer crosses the gelation point and nanoparticles form.

Because the droplet size and spatial distribution of strands of recombinant polymer within the liquid droplet are not necessarily monodisperse and uniform, respectively, nanoparticles will form with a distribution of diameters. The next step involves separating the nanoparticles based on size to produce nanoparticles that are substantially uniform in size. In one aspect, a differential mobility analyzer (DMA) can be used to purify and dramatically increase the uniformity of the nanoparticles. The combined electrospray differential mobility analysis system used herein is termed “ES-DMA.” ES-DMA is conceptually similar to mass spectrometry (MS), but it differs from ES-MS in several ways. First, because the DMA operates at atmospheric pressure, the nanoparticles and encapsulated bioactive agent are subject to aerodynamic drag. The instrument, thereby, separates them based on their charge-to-aerodynamic diameter ratio (i.e., aerosol electrical mobility) as opposed to the charge-to-mass ratio. Second, electrosprayed droplets pass through a neutralizing chamber to reduce the charge on each to +1, 0, or −1. Thus, the effective diameter of the particle may be determined directly by dividing the charge by the charge-to-diameter ratio. Third, ES-DMA can characterize species with molecular weights greatly exceeding 10 kDa, which makes it well suited for characterizing larger nanoparticles useful as drug delivery devices.

Although ES-DMA is one approach to separating the nanoparticles based on size to produce nanoparticles that are substantially uniform in size, other techniques can be used herein. For example, separation techniques including, but not limited to centrifugation and field flow fractionation can be used

The methods described herein can produce nanoparticles that are substantially uniform in size. Synthetic delivery systems such as polymers have the potential to reduce the safety problems associated with delivery of bioactive agents. Polymeric carriers synthesized using traditional chemical synthetic methods result in polymers with random sequences, distribution of molecular weights, and difficulty in attaching functional motifs at precise locations. Furthermore, nanoparticles composed of these polymers are further heterogeneous both in diameter and in the number of strands that compose an individual particle. The methods described herein permit the high level of simultaneous control over both nanoparticle size and polymer composition, which makes the nanoparticles described herein very useful in developing fine-tuned delivery devices for bioactive agents.

In one aspect, the uniformity of the particle size can be quantified by determining the coefficient of variation of the nanoparticles. The “coefficient of variation” is defined as the ratio of the standard deviation to the mean in terms of either diameter or radius. The mean diameter can be determined as the peak particle size or the number average particle size as measured in by the DMA. Formulas for determining the width of the particle size distribution, which should be very similar, if not identical, to the width of the deposited nanoparticle distribution are known in the art. (See Stolzenburg, M. R. An Ultrafine Aerosol Size Distribution Measuring System. Ph.D Thesis. University of Minnesota, Minneapolis, 1988; and Stolzenburg, M. R.; McMurry, P. H., Equations Governing Single and Tandem DMA Configurations and a New Lognormal Approximation to the Transfer Function. Aerosol Science and Technology 2008, 42, 421-432). One measure of the width is the standard deviation. In the Examples below, the mean particle diameter and standard deviation were calculated as follows. The nanoparticle diameter was determined by measuring the observed perimeter and dividing it by π (i.e., 3.1415 . . . ). Alternatively, the diameter as the length from one end to the other through the particle center can be used as well. Both approaches can be used herein. A histogram of diameters was assembled. The average diameter was measured in this case from at least 200 nanoparticles. The standard deviation was also calculated from this population of diameters. The standard deviation was then divided by the mean to determine the coefficients of variation as reported.

In one aspect, the coefficient of variation is less than 15%, less than 10%, or less than 5%. In other aspects, the coefficient of variation is from 0.5% to 10%, 1% to 9%, 2% to 8%, 3% to 6%, or 3.5% to 5%. As discussed above, the size of the nanoparticles produced herein can vary depending upon reaction and solution conditions. In one aspect, the nanoparticles have a diameter from 1 nm to 250 nm, 5 nm to 200 nm, 10 nm to 100 nm, or from 10 nm to 60 nm. The size of the nanoparticles can be selected depending upon the application of the nanoparticles. Additionally, the shape of the nanoparticles can vary as well. For example, the nanoparticles can be spherical or cylindrical. Not wishing to be bound by theory, the shape of the nanoparticle can influence the ability of the nanoparticles to effectively deliver a bioactive agent to a specific target. The Examples discuss in detail the conditions for producing nanoparticles with different shapes in a controlled manner.

### III. Pharmaceutical Compositions

The nanoparticles described herein can be administered to a subject using techniques known in the art. For example, pharmaceutical compositions can be prepared with the nanoparticles. It will be appreciated that the actual preferred amounts of the nanoparticles with the bioactive agent in a specified case will vary according to the specific compound being utilized, the particular compositions formulated, the mode of application, and the particular site and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity-enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

Parenteral vehicles (e.g., intravenous, intramuscular, or subcutaneous), if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

### IV. Methods of Use

The nanoparticles described herein can be used to introduce a bioactive agent into a target cell. The method generally involves contacting the target cell with the nanoparticle, wherein the bioactive agent is taken up by the target cell. In one aspect, the nanoparticles described herein can facilitate the delivery of nucleic acids as therapy for genetic disease by supplying deficient or absent gene products to treat any genetic disease or by silencing gene expression. Techniques known in the art can be used to measure the efficiency of the compounds described herein to deliver nucleic acids to a cell.

In other aspects, the target cell comprises stem cells, committed stem cells, differentiated cells, primary cells, and tumor cells. Examples of stem cells include, but are not limited to, embryonic stem cells, bone marrow stem cells and umbilical cord stem cells. Other examples of cells used in various embodiments include, but are not limited to, osteoblasts, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, and neurons.

The nanoparticles described herein are effective in delivering bioactive agents into cells, which can ultimately be used to treat or prevent a number of different diseases. The term “treat” as used herein is defined as reducing the symptoms of the disease or maintaining the symptoms so that the symptoms of the disease do not become progressively worse. The term “treat” is also defined as the prevention of any symptoms associated with the particular disease. The term “effective amount” as used herein is the amount of nanoparticles and bioactive agent sufficient to treat the disease in the subject upon administration to the subject. In one aspect, the nanoparticles described herein can be used as carriers to deliver bioactive agents (e.g., nucleic acids) to treat or prevent cancer in a subject. Examples of different types of cancers include, but are not limited to, breast cancer, liver cancer, stomach cancer, colon cancer, pancreatic cancer, ovarian cancer, lung cancer, kidney cancer, prostate cancer, testicular cancer, glioblastoma, sarcoma, bone cancer, head-and-neck cancers, and skin cancer.

Due to the ability of the methods described herein to produce nanoparticles that are substantially uniform in size as well as composed of recombinant polymers that are uniform in size, sequence, and structure, the nanoparticles can be synthesized in a manner in which they are very specific in function and performance. In other words, the nanoparticles can be fine tuned to target specific cells. Additionally, the methods described herein permit the formation of nanoparticles that have specific biodegradation patterns. Thus, the nanoparticles can be designed in a manner such that the nanoparticles deliver the bioactive agent in a controlled manner.

In other aspects, the nanoparticles described herein can be used to image a cell or tissue in a subject. In one aspect, the method comprises (1) administering to the subject the nanoparticles described herein composed of an imaging agent, and (2) detecting the imaging agent. The methods can be used to image healthy cells and tumor cells. A variety of different tissues and organs can be imaged using the methods described herein including, but not limited to, liver, spleen, heart, kidney, lung, esophagus, bone marrow, lymph node, lymph vessels, nervous system, brain, spinal cord, blood capillaries, stomach, ovaries, pancreas, small intestine, and large intestine. Techniques known in the art for detecting the imaging agent once incorporated into the cells or tissue are known in the art. For example, magnetic resonance imaging (MRI) can be used to detect the imaging agent. Additionally, the nanoparticles described can be indispensable tools in a variety of other medical procedures, including, but not limited to, angiography, plethysmography, lymphography, mammography, cancer diagnosis, and functional and dynamic MRI.

In other aspects, nanoparticles with pharmaceutical agents and imaging agents can be administered to a subject concurrently or sequentially. For example, a first nanoparticle composed of a pharmaceutical compound can be admixed with a second nanoparticle composed of an imaging agent. The first and second nanoparticles can be the same or different. It is also contemplated that a mixture of two or more different nanoparticles with the same or different bioactive agent or imaging agent can be administered to the subject.

It is understood that any given particular aspect of the disclosed compositions and methods can be easily compared to the specific examples and embodiments disclosed herein. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

The three SELPs mentioned in Table 1 were biosynthesized as described in Dandu, R., Cresce, V. A., Briber, R., Dowell, P., Cappello, J., and Ghandehari, H., Silk-elastinlike Protein Polymer Hydrogels: Influence of Monomer Sequence on Physicochemical Properties. Polymer 2009, 50, 366-374. The polymers were stored at −80° C. until diluted in ammonium acetate buffer to the concentrations listed in the figure captions.

TABLE 1
Composition of silk-elastinlike protein polymers.

| | Silk | Elastin | Silk-Elastin | Mol. Wt |
|---|---|---|---|---|
| Polymer* | Units | Units | Blocks/Strand | (kDa) |
| SELP-47K | 4 | 8 | 13 | 69.8 |
| SELP-415K | 4 | 16 | 8 | 71.5 |
| SELP-815K | 8 | 16 | 6 | 65.3 |

Nanoparticles were fabricated using an electrospray (ES) droplet evaporation technique. An ES aerosol generator (TSI 3480) uses pressure driven flow through a 25 micron capillary. At the tip of the capillary, a voltage was applied to form a Taylor cone-jet. Droplets emitted from the jet were entrained in a mixed stream of air and carbon dioxide at atmospheric pressure. Typical droplets spanned 150 nm to 300 nm in diameter though larger sizes were possible. Several polymer strands may have resided within each droplet. As the jet broke up into droplets they passed through a charge neutralizer (Po-210) to reduce the net charge on the aerosolized droplet to a single net charge. In the charge neutralizer, as solvent evaporated, the droplet dried and polymeric nanoparticles were formed. Because the droplet size and spatial distribution of strands within the liquid droplet were not necessarily monodisperse and uniform, nanoparticles formed with a distribution of diameters. They entered a differential mobility analyzer (DMA-TSI 3085) where they were purified based on their charge-to-size ratio. Voltage was supplied to the nano-DMA through a high voltage supply (BERTAN 205B-10R).

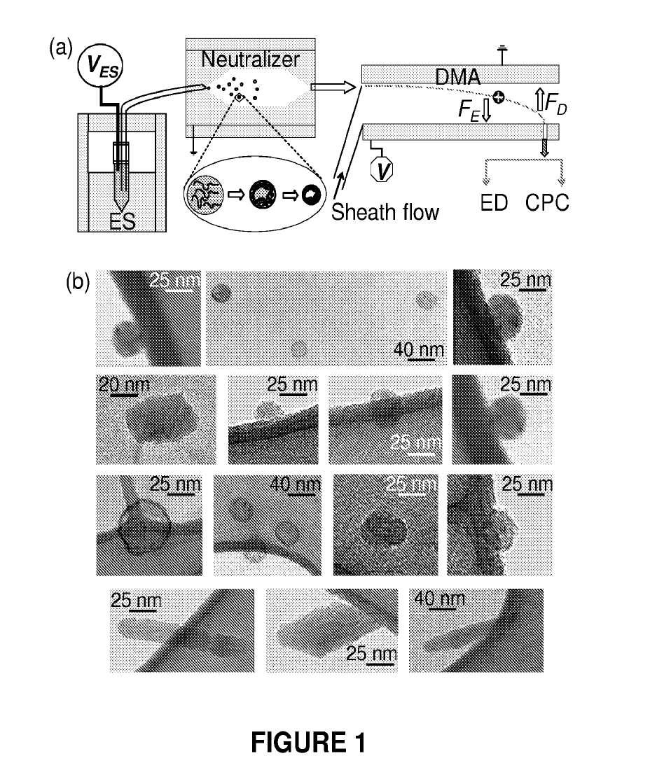

Within the DMA an aerosolized particle in an electric field, Eₛ, carrying nₑ electric charges experienced an electrostatic force dragging it towards the electrode. The particle very quickly reached its terminal velocity, v, and the electrostatic force acting on the particle was balanced by a resulting drag force on the particle, given by Stokes Law, which determines the electrical mobility of a particle as shown in FIG. 1a Combining particle mobility and instrument mobility determines the mobility diameter, dₚ of the particle and is given by

$$\frac{{d}_{p}}{{C}_{c}} = \frac{2 {n}_{e} e VL}{3 {μ}_{g} {q}_{sh} ln \frac{{r}_{2}}{{r}_{1} }} , \qquad ( 4 )$$

where, C_{c} is Cunningham slip correction factor, e is elementary charge on the particle (1.6×10⁻¹⁹ C), V is average voltage on the collector rod inside the DMA, qₛₕ is sheath flow (nitrogen), μ_{g} is gas viscosity, L is length between polydisperse aerosol inlet and exit slit (4.987 cm), r₁ is inner radius of annular space of the DMA (0.937 cm), and r₂ is outer radius of annular space of the DMA (1.905 cm).

Monodispersed aerosol produced from the DMA was measured in concentration through an ultrafine condensation particle counter (CPC-TSI 3776) by collecting data for 17 seconds with a 3 second interval between each measurement or was deposited on a desired substrate (TEDPELLA 01824, in the present case) for TEM imaging using a nanometer aerosol sampler (TSI 3089) which has an electric field that collects charged particles from the inlet onto a portion of the substrate. FIG. 1a shows a schematic of this set-up. The combined electrospray differential mobility analysis system is termed as ES-DMA or gas-phase electrophoretic mobility molecular analysis (GEMMA).

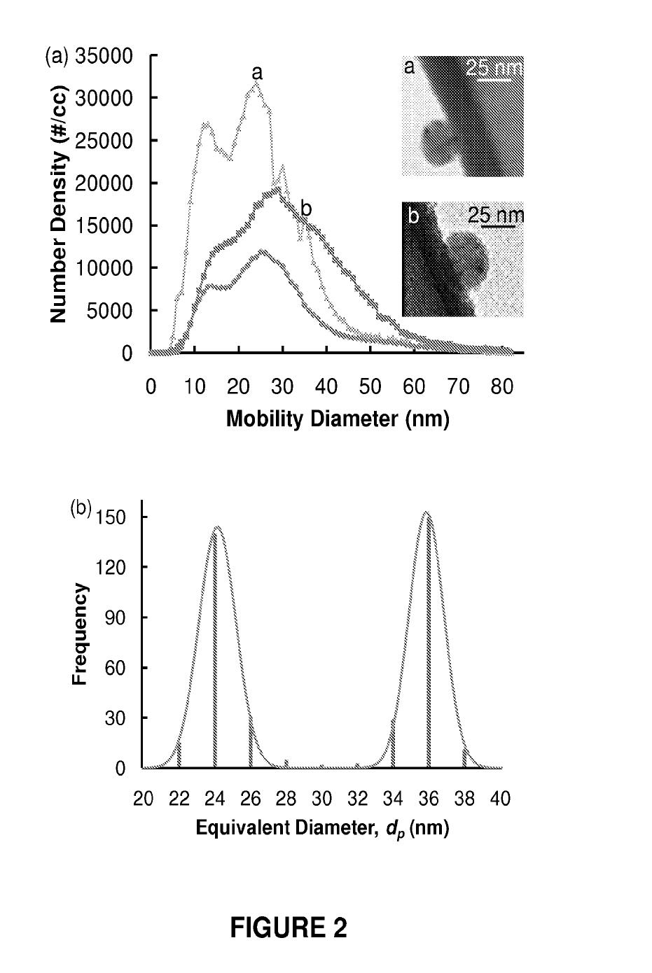

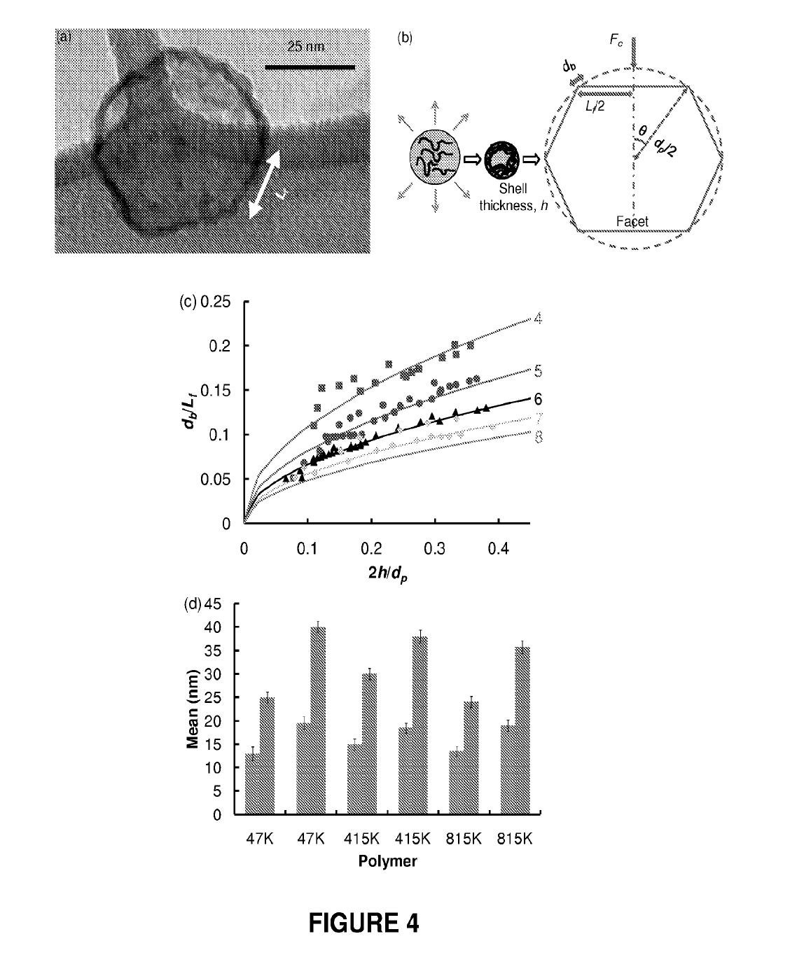

A transmission electron microscope (TEM), a FEI Tecnai T-12, was used at high tension (120 kV) to obtain the images of the polymeric nanoparticles. TEM microscopy revealed that these particles have several facets (edges, explained above) and the equivalent diameter was approximated from the TEM images as, dₚΣᵢ=which is exact for a sphere, where, L_{f}ⁱ is the length of facet i (see FIG. 4a). This formula is used to determine the mean and standard deviation for calculating the coefficient of variation. Histograms were constructed for dₚ and L_{f} (FIG. 2b and FIG. 4d) using a mid-point labeled bin.

Sugar solutions in ammonium acetate (2 mM) were used to evaluate the size of the electrospray droplets. A mass balance between the droplet and the sugar particle yields a relation that the droplet diameter (d_{drop}) is ˜19 times of the particle diameter (dₚ) for the conditions reported herein. A 20 mmol/L ammonium acetate solution (pH ˜8) was prepared using milli-Q water, purified by milli-Q integrated water purification system from Millipore, Inc. Acetic acid and ammonium hydroxide were used to adjust the pH to ˜8. Milli-Q water was used for dilution to obtain the reported buffer concentrations. This ensures negligible contribution of non-volatile salts from the buffer.

The diameter of the jet is a strong function of the viscosity of the solution and the viscosity of all the three polymers under experimental conditions was measured using a calibrated semi-micro viscometer (Cannon-Manning 9722-D50). A pipette gun was used to apply the suction pressure on the glass capillary tube to ensure that humidity did not affect the viscosity measurements. Table 2 gives the values of dynamic viscosity of polymers at different concentrations.

TABLE 2
Dynamic viscosities of SELPs at different weight concentrations.

| | Concentration | Viscosity |
|---|---|---|
| Polymer | (wt. fraction) | (mPa · s) |
| SELP-47K | 1.33 · 10⁻³ | 1.23 |
| | 0.88 · 10⁻³ | 1.19 |
| | 0.66 · 10⁻³ | 1.17 |
| | 0.44 · 10⁻³ | 1.13 |
| SELP-415K | 1.33 · 10⁻³ | 1.30 |
| | 0.88 · 10⁻³ | 1.24 |
| | 0.66 · 10⁻³ | 1.15 |
| | 0.44 · 10⁻³ | 1.09 |
| SELP-815K | 1.33 · 10⁻³ | 1.26 |
| | 0.88 · 10⁻³ | 1.22 |
| | 0.66 · 10⁻³ | 1.18 |
| | 0.44 · 10⁻³ | 1.12 |

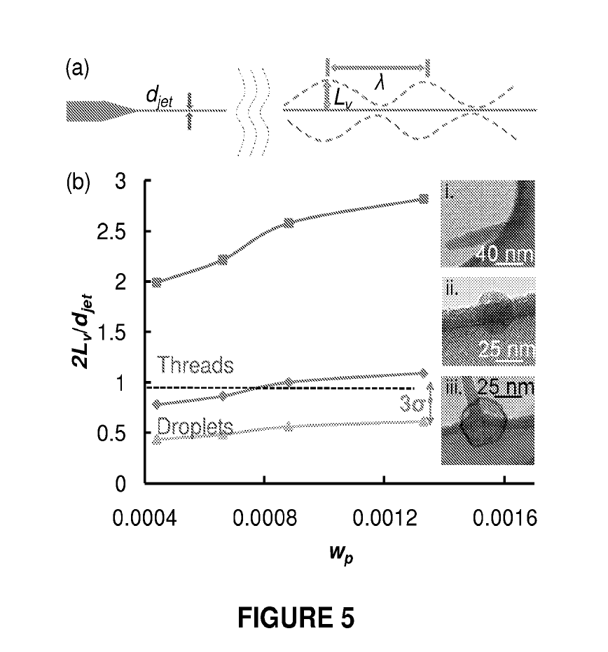

Capillary forces, viscous force, and electrical stresses characterize the growth rate of perturbation and are given by the dimensionless numbers, wave number, k′=πdⱼₑₜ/λ, which captures the disturbance frequency, ratio of electric stress over surface tension stress, R_{ES}=σ_{c}²dⱼₑₜ/4γε₀, and Ohnesorge number, Oh=μₗ(γρdⱼₑₜ)^{1/2}, which captures the ratio of viscous forces to capillary forces. Here, dⱼₑₜ is the diameter of the jet, σ_{c} is the surface charge density, and ε₀ is the permittivity of the vacuum. The perturbation growth rate is a function of dⱼₑₜ and λ (see FIG. 5a) calculated using the dispersion relation,

$${ω}^{′ 2} + {2}^{1 / 2} Oh {μ}_{l} {k}^{′ 2} \frac{24 + {k}^{′ 2}}{8 + {k}^{′ 2}} {ω}^{′} = \frac{4 {k}^{′ 2}}{8 + {k}^{′ 2}} ( 1 - {k}^{′2} - 2 RES ( 1 + {k}^{′} ( Limit ( m -> 0 ) \frac{{I}_{m}^{′} ( {k}^{′} ) - {I}_{- m}^{′} ( {k}^{′} )}{{I}_{m} ( {k}^{′} ) - {I}_{- m} ( {k}^{′} )} ) ) ) , \qquad ( 5 )$$

and a mass balance on the jet and droplet,

$${d}_{jet} = {( \frac{2 {d}_{drop}^{3}}{3 λ} )}^{1 / 2} . \qquad ( 6 )$$

The jet break up phenomenon occurred at the maximum growth rate and this jet diameter depended on the droplet diameter. Reasonable initial guesses for dⱼₑₜ and λ, yields the maximum growth rate and a corresponding dimensionless wave number, k′. k′ gives a new characteristic period λ_{new} and using this value, Eq. 6 gives a new jet diameter, dⱼₑₜ^{new} for a particular droplet diameter (100-300 nm as in FIG. 5b). The maximum growth rate is calculated for these new set of values and iterating until 1-dⱼₑₜ^{new}/dⱼₑₜ^{old}≦10⁻⁴, yields dⱼₑₜ, for different droplet diameters (FIG. 5b). When the ratio, 2Lᵥ/dⱼₑₜ<1 the jet breaks up into droplets and when the ratio is >1, threads are formed.⁴¹ For our system, the values of dimensionless numbers are, k′=0.494-1.819, Rₑₛ=0.404-1.85, and Oh=0.466-1.18.

### Results and Discussion

These highly uniform nanoparticles form from electrospray droplets that capture multiple polymer strands (see FIG. 1a). Evaporation ensues, simultaneously shrinking the droplet diameter and leading to accumulation of polymer at the droplet interface. The polymer forms a thin film or shell around the exterior of the droplet. Further evaporation compresses the shell and concentrates the polymer remaining in the core. FIG. 1b shows a gallery of representative transmission electron microscopy (TEM) images of the nanoparticles thus formed. Most of the nanoparticles are approximately spherical and display modest faceting, though some are elongated with sharp facets. FIG. 2 shows that these nanoparticles are also heterogeneous in size with diameters ranging from 5 nm to over 60 nm, which is not atypical of nanoparticles formed from traditional particles. The nanoparticles were then purified by size using a DMA (see FIG. 1a).

The prominent feature of the recombinant nanoparticles is their uniformity after size purification through the DMA. The width and mean of the distribution of diameters depends on several factors including the polymer composition. The three SELPs selected have approximately equal molecular weights ranging from 66 kDa to 71 kDa (see Table 1) but distinct silk-to-elastin ratios of approximately ½ for SELP-47K and SELP-815K and ¼ for SELP-415K. FIG. 2a suggests that decreasing this ratio leads to wider distributions. However, following size separation using the DMA, the distributions narrow dramatically. The insets to FIG. 2a show nanoparticles collected at two sizes, while FIG. 2b shows two histograms of the nanoparticle diameter each assembled from nearly 200 TEM images of nominally 24.0 nm or 36.0 nm SELP particles collected at the indicated positions in FIG. 2a.

Statistical compilation and Gaussian curves in FIG. 2b show that the standard deviation on the size of these DMA selected particles is 1.2 nm and 1.4 nm for the nominally 24.0 nm and 36.0 nm sizes, respectively. A Gaussian distribution is not unexpected because Stolzenberg indicates that diffusional broadening within the DMA contributes to instrument uncertainty and follows this distribution. In net, this leads to coefficients of variation of <5%, which is equal to or better than those reported for metallic nanoparticles and rivals that of biologically assembled particles such as viruses. These results demonstrate that ES-DMA can both generate and purify polymeric nanoparticles with high dimensional uniformity.

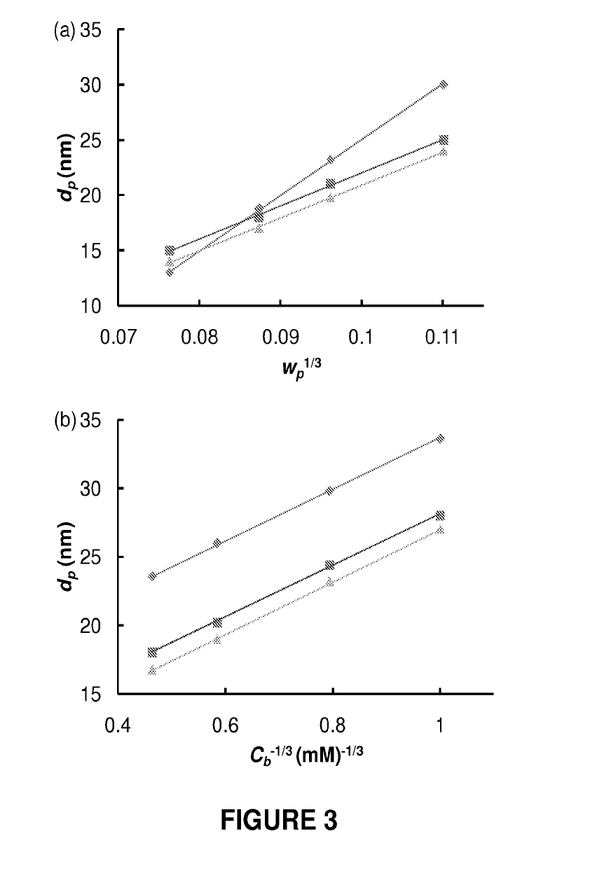

The size distributions also depend on the polymer concentration or weight fraction, wₚ and buffer concentration, C_{b}. These two parameters are important because they provide the ability to tune the yield of particles selected by the DMA by positioning the peak maximum at the diameter selected for size purification. FIG. 3a shows that increasing wₚ leads to larger nanoparticles with a power law dependence of dₚ˜wₚ^{1/3}. Conversely, FIG. 3b shows that increasing C_{b} leads to smaller nanoparticles with a power law dependence of dₚ˜C_{b}^{-1/3}.

The exponents in FIG. 3 follow directly by comparing the polymer mass contained in the droplet before evaporation (wₚρ_{w}πd_{drop}³/6) and the nanoparticle after evaporation (ρₚπdₚ³/6). Equating the two masses gives an expression for the particle diameter, dₚ=(ρ_{w}/ρₚ)^{1/3}d_{drop}wₚ^{1/3}, where ρ_{w} and ρₚ are densities of polymer in droplet and particle, respectively, and d_{drop} is the diameter of the electrospray droplet. Because polymer concentration in the droplet is initially very modest, the density of the droplet is essentially that of pure water. The mass balance immediately explains the dependence of the particle diameter on the weight fraction of the polymer in FIG. 3a. It was found experimentally that κ depends linearly on C_{b}. Substituting C_{b} for κ yields dₚ˜C_{b}^{-1/3} in excellent agreement with FIG. 3b.

FIG. 3a illustrates that SELP-415K nanoparticles have a different diameter than SELP-47K and -815K nanoparticles, depending on the polymer concentration. As the droplet evaporates, polymer strands accumulate at the interface forming a shell. When the crosslinking reaction is slower than the evaporation rate, silk and elastin units in the SELPs start to crosslink after the shell has formed. After complete evaporation, compression forces are released, resulting in expansion of the elastin units. The percentage of crosslinkable units in SELP-47K and -815K is similar to one another and approximately double that of 415K. This lower crosslinking density of SELP-415K allows the elastin units to expand more, yielding a particle with larger diameter and thinner shell. However, particles of SELP-47K and -815K remain smaller due to more crosslinking. This prediction of thinner shells in SELP-415K is supported by the shell thickness measurements, which show that SELP-415K, -47K, and -815K have an average shell thickness of 4.8±1.4 nm, 6.0±0.8 nm, and 6.2±0.8 nm, respectively (sample size=32 shells), for particle sizes of 29 nm, 25 nm, and 24 nm. The difference in the average shell thicknesses of SELP-47K and -415K is significant at a 99% confidence level based on student's t test whereas that of SELP-47K and -815K is not significant. These observations are also consistent with the physicochemical properties of these polymers investigated previously where the modulus of elasticity of SELP-415K is lower than that of SELP-47K and SELP-815K.

Surprisingly, several of the SELP particles in FIG. 1a are facetted or display nearly straight edges as magnified in FIG. 4a. TEM diffraction studies found no indication of ordering, suggesting that crystallization of the polymer was not responsible for the faceting. However, most of the TEM images suggest an increased density of the polymer on the SELP particle perimeter and these edges are not sharp as expected of crystallization, leading to the hypothesis that a buckling instability may be responsible for the apparent faceting. In this scenario, electrospray droplets, consisting of SELP, water, and ammonium acetate selected for its volatility, immediately begin to dry. As the solvent evaporates, the polymer strands accumulate at the air-water surface and tangle or gel into a thin film or shell. Further evaporation compresses the shell, developing compression stresses that the entangled strands cannot completely relax by shrinking the particle perimeter. As more solvent evaporates through the shell, its compression energy increases further until it becomes energetically favorable for the shell to bend to relieve compression energy. FIG. 4b shows a diagram of this process. Landau and Liftshitz show that the compression and bending energies, E_{c} and E_{b}, scale as

$${E}_{c} = \frac{2 Eh {δ}^{2}}{{d}_{p}^{2}} {L}_{f} {d}_{b} and {E}_{b} = \frac{{Eh}^{3} {δ}^{2} {L}_{f}}{2 {d}_{b}^{3}} , \qquad ( 1 )$$

where E is elastic modulus, h is shell thickness, δ is the displacement of points on the shell from an ideal sphere in the bending strip defined as θd_{b} (see FIG. 4b), and L_{f} represents the facet length of a SELP nanoparticle (see FIG. 4a) (Landau, L. D.; Lifshitz, E. M., Theory of Elasticity. Pergamon Press 1959, 7, 62-65). In the neighborhood of a bend, the local bending diameter representing the local curvature of the particle is given as d_{b}, and the diameter of the particle is estimated by summing the lengths of each facet and dividing by π, such that dₚ=ΣᵢL_{f}ⁱ/π. The sum of these two energies may be minimized with respect to d_{b} to find d_{b}=3^{-1/4}h^{1/2}dₚ^{1/2}2^{-1/2}. We scale d_{b} on L_{f} such that this dimensionless ratio varies between zero and unity and substitute L_{f}=dₚ Sin θ, where θ is related to the number of sides or facets, n, by θ32 π/n. Then,

$$\frac{{d}_{b}}{{L}_{f}} = \frac{{3}^{1 / 4}}{2} {( \frac{2 h}{{d}_{p}} )}^{1 / 2} \frac{1}{Sin ( π / n )} . \qquad ( 2 )$$

FIG. 4c shows the relationship between these dimensionless ratios for n ranging from 4 to 8. Each parameter in Eq. 2 can be estimated experimentally from TEM images (like FIG. 4a) for SELP nanoparticles. Comparing experiment to theory shows good quantitative agreement, confirming the hypothesis that a buckling instability governs facet formation. Remarkably, FIG. 4c also indicates that the nanoparticles are essentially hollow with the shell comprising 10 to 40% of the particle radius. This can be confirmed by a mass balance where the nanoparticle volume is given by ρₚπ[dₚ³−(dₚ−h) ³]/6 such that

$$h = \frac{{d}_{p}}{2} [ 1 - {w}_{p} \frac{{ρ}_{w}}{{ρ}_{p}} \frac{{d}_{drop}^{3}}{{d}_{p}^{3}} ] . \qquad ( 3 )$$

Evaluating the data points in FIG. 3 using Eq. 3 also leads to the conclusion that the nanoparticles are hollow.

Experimentally, it was found that SELP nanoparticles possess 4 to 7 facets with the preponderance having 5 or 6, indicating that dₚ/L_{f} should be 2.00±0.30 from the particle geometry considerations in FIG. 4b. This ratio can also be determined from 200 nanoparticles captured in TEM images for each of the three polymers at two sizes (25.0 and 39.0 nm for SELP-47K, 29.0 and 38.0 nm for SELP-415K, and 24.0 and 36.0 nm for SELP-815K) with smaller sizes corresponding to peak maxima.

FIG. 4d shows the compiled means and standard deviations (error bars represent 1σ) for L_{f} and dₚ. Comparing the particle diameter and facet length finds dₚ/L_{f}=1.95±0.41, which is also in good agreement with theory. Notably the uncertainty in the nanoparticle size remains uniform regardless of diameter in this size range.

FIG. 1b also shows several of the nanoparticles to be elongated and rod-shaped (see bottom row). These particles form when the electrospray instability that leads to droplet formation is suppressed. At the exit of the electrospray capillary (see FIG. 1a) large electric fields lead to the formation of a Taylor cone, from which a narrow jet emerges. As the jet evolves from the tip of the capillary, a varicose or symmetric perturbation grows on the surface of the jet characterized by a differential radius, Lᵥ, as depicted in FIG. 5a. Eggers and Christiani, et al. indicate that the jet breaks up into droplets when 2Lᵥ/dⱼₑₜ remains less than unity but remains as polymeric filaments when this ratio exceeds unity (Christanti, Y.; Walker, L. M., Surface Tension Driven Jet Breakup of Strain-Hardening Polymer Solutions. J. Non-Newtonian Fluid Mech. 2001, 100, 9-26; Eggers, J., Universal Pinching of 3D Axisymmetric Free-surface Flow. Phys Rev Lett 1993, 71, 3458-3460). The numerator is given by Eggers as Lᵥ=μₗ²/ργ, where μₗ, ρ, and γ are the dynamic viscosity, density, and surface tension of the polymer solution. However, dⱼₑₜ is not known a priori but must be inferred from the model of Christianti as described in the methods section. The ratio depends on both the polymer concentration and the conductivity of the electrospray solution as shown in FIG. 5b. Either increases the probability of thread formation.

First, increasing the polymer weight fraction to 0.0025 exclusively produces long strands that can be observed visually at the tip of the Taylor cone. Second, depositing all particles emerging from the electrospray at wₚ=0.0013 and C_{b}=2 mM finds a minority of particles to be rod shaped commensurate with the uncertainty in dⱼₑₜ (see FIG. S3 in Supporting Information). The ability to select for or against rod like particles is important since it has recently been shown that the shape of nanoparticles can influence biodistribution and cellular uptake.

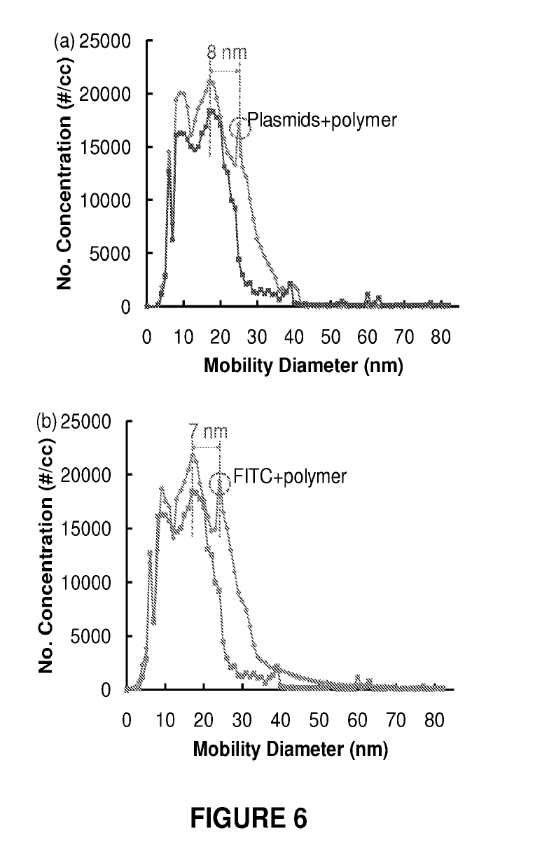

Finally, it was demonstrated that these highly uniform nanoparticles may be developed into carriers of therapeutic agents. The advantages of doing so are not only in the precision of the nanoparticle and tunability of the polymer properties, but the ease with which therapeutic agents can be incorporated within these particles. Simply including the therapeutic agent in the polymer solution to be electrosprayed will lead to incorporation within the nanoparticle. For example, SELP-815K, a polymer shown to maximize duration and extent of gene expression, was mixed with plasmid DNA and fluorescein isothiocyanate (FITC) in FIG. 6a and FIG. 6b respectively.

In both cases a new peak arises 7-8 nm from the primary peak and the distribution of all particles is wider. The new peaks in the size distributions are remarkably repeatable and strongly indicate the incorporation of these model agents of gene and drug delivery into the SELP nanoparticles.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. Nanoparticles produced by the process comprising:
a. providing a solution comprising one or more recombinant polymer in a solvent;
b. forming droplets comprising the one or more recombinant polymers and the solvent;
c. removing the solvent to produce the nanoparticles; and
d. separating the nanoparticles based on size to produce nanoparticles that are substantially uniform in size.

2. The nanoparticles of claim 1, wherein the recombinant polymer comprises silk-like units, elastin-like units, collagen-like units, keratin-like units, or any combination thereof.

3. The nanoparticles of claim 1, wherein the polymer comprises one or more silk-elastinlike protein polymer (SELP).

4. The nanoparticles of claim 3, wherein the ratio of silk-like units to elastin-like units present in the SELP is from 1:20 to 20:1.

5. The nanoparticles of claim 3, wherein the ratio of silk-like units to elastin-like units present in the SELP is from 1:2 to 1:4.

6. The nanoparticles of claim 3, wherein SELP has a molecular weight from 25 kDa to 200,000 kDa.

7. The nanoparticles of claim 1, wherein the polymer comprises a residue of at least one cationic amino acid.

8. The nanoparticles of claim 1, wherein the polymer comprises SEQ ID NO 1, SEQ ID NO 2, SEQ ID NO 3, or any combination thereof.

9. The nanoparticles of claim 1, wherein the nanoparticles have a diameter from 1 nm to 250 nm.

10. The nanoparticles of claim 1, wherein the nanoparticles have a diameter from 10 nm to 60 nm.

11. The nanoparticles of claim 1, wherein the nanoparticles have a coefficient of variation of less than or equal to 15%.

12. The nanoparticles of claim 1, wherein the solution of the polymer further comprises a bioactive agent.

13. The nanoparticles of claim 12, wherein the bioactive agent comprises a natural or synthetic oligonucleotide, a natural or modified/blocked nucleotide/nucleoside, a nucleic acid, a peptide comprising natural or modified/blocked amino acid, an antibody or fragment thereof, a virus, a hapten, a biological ligand, a membrane protein, a lipid membrane, an imaging agent, or a small pharmaceutical molecule.

14. The nanoparticles of claim 12, wherein the bioactive agent comprises DNA or a fragment thereof.

15. The nanoparticle of claim 12, wherein the bioactive agent comprises RNA or a fragment thereof.

16. The nanoparticles of claim 1, wherein polymer further comprises a protein or peptide targeting ligand.

17. The nanoparticles of claim 1, wherein polymer further comprises a nuclear localization sequence, an endosome disrupting moiety, or a combination thereof.

18. The nanoparticles of claim 1, wherein the solution comprises water or a buffered aqueous solution.

19. The nanoparticles of claim 1, wherein the droplets are formed by an electrospray aerosol generator or a nebulizer.

20. The nanoparticles of claim 1, wherein step (c) comprises passing the droplets through a charge neutralizer.

21. The nanoparticle of claim 1, wherein step (d) comprises introducing the nanoparticles through a differential mobility analyzer, wherein the nanoparticles are purified based on charge-to-size ratio.

22. Nanoparticles comprising one or more recombinant polymers and a bioactive agent, wherein the nanoparticles have a coefficient of variation of less than or equal to 15%.

23. The nanoparticles of claim 22, wherein the recombinant polymer comprises a silk-elastinlike protein polymer (SELP) and the bioactive agent comprises a nucleic acid.

24. A pharmaceutical composition comprising the nanoparticles of claim 1 and a pharmaceutically acceptable carrier.

25. A method for systemically delivering a bioactive agent to a subject, the method comprising administering to the subject nanoparticles comprising one or more recombinant polymers and a bioactive agent, wherein the nanoparticles are substantially uniform in size.

26. The method of claim 25, wherein the nanoparticles are injected parenterally into the subject.

27. The method of claim 25, wherein the nanoparticles are administered intravenously, intramuscularly, or subcutaneously to the subject.

28. A method for treating cancer in a subject, the method comprising administering to the subject nanoparticles comprising one or more recombinant polymers and a bioactive agent in an effective amount to treat the cancer, wherein the nanoparticles are substantially uniform in size.

29. The method of claim 28, wherein the cancer comprises breast cancer, liver cancer, stomach cancer, colon cancer, pancreatic cancer, ovarian cancer, lung cancer, kidney cancer, prostate cancer, testicular cancer, glioblastoma, sarcoma, bone cancer, head-and-neck cancers, and skin cancer.

30. A method for delivering a bioactive agent to a target cell, the method comprising contacting the target cell with nanoparticles comprising one or more recombinant polymers and a bioactive agent, wherein the nanoparticles are substantially uniform in size.

31. A method for imaging a cell or tissue in a subject comprising (1) administering to the subject a nanoparticle of claim 1 comprising an imaging agent, and (2) detecting the imaging agent.
